(19) United States
(12) Patent Application Publication
(10) Pub. No.: US 2025/0143811 A1
(43) Pub. Date: May 8, 2025

(54) REUSABLE REGISTRATION KIT FOR SPINAL ROBOTICS

(71) Applicant: Mazor Robotics Ltd., Caesarea (IL)

(72) Inventors: Zhinan Guo, Shanghai (CN); Jiangfeng Zhang, Shanghai (CN)

(21) Appl. No.: 18/835,987

(22) PCT Filed: Feb. 15, 2022
(86) PCT No.: PCT/CN2022/076278

§ 371 Date: Aug. 5, 2024

(51) Int. Cl. A61B 34/30

(52) U.S. Cl. CPC A61B 34/30

(57) ABSTRACT

A robotic registration kit according to at least one embodiment of the present disclosure includes an elongate arm (304) extending from a proximal end (302) to a distal end (306) including a target receiving surface (308) and a robot mount bracket (312) including a robot attachment surface (358), a clamp screw (316), including a bracket contact surface in contact with the robot mount bracket (312) and a threaded outer surface (322), rotationally attached to the robot mount bracket (312) allowing the clamp screw (316) to rotate and preventing axial movement of the clamp screw (316), and a clamp (320) including a plate (324) with a threaded hole (328) running therethrough and a finger (332) extending a distance to a clamp end including a hook portion (336) disposed proximally past the robot attachment surface (358) of the robot mount bracket (312). The clamp (320) is moveable between a clamped state and an unclamped state by a rotational movement of the clamp screw (316) relative to the clamp (320).

### BACKGROUND

The present disclosure is generally directed to surgical systems, and relates more particularly to registration kits for robotic surgical devices.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures. Prior to use, spatial registration of the surgical robot ensures that movements of the linked articulating members are calibrated.

BRIEF SUMMARY

Example aspects of the present disclosure include:

A robotic registration kit according to at least one embodiment of the present disclosure comprises: an elongate arm extending longitudinally from a proximal end to a distal end, the elongate arm comprising: a target receiving surface disposed at the distal end; and a robot mount bracket disposed at the proximal end and protruding from the elongate arm in a direction away from a longitudinal axis of the elongate arm, the robot mount bracket comprising a robot attachment surface disposed at a proximal end of the robot mount bracket; a clamp screw extending a length along a rotation axis from a first end of the clamp screw to a second end of the clamp screw, the clamp screw comprising: a bracket contact surface disposed at the first end, wherein the bracket contact surface is in contact with the robot mount bracket; and a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis; and a clamp, comprising: a plate; a threaded hole running through the plate, wherein the threaded outer surface of the clamp screw is threadedly engaged with the threaded hole; and a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp.

Any of the aspects herein, wherein a registration target frame is mounted to the target receiving surface of the elongate arm, the registration target frame comprising marker beads that are arranged in a pattern on at least one surface of the registration target frame.

Any of the aspects herein, wherein a sterile sleeve fully encloses the robotic registration kit maintaining an environment inside the sterile sleeve from an environment outside the sterile sleeve.

Any of the aspects herein, wherein the sterile sleeve comprises a thin film bag sealed around the robotic registration kit, and wherein the sterile sleeve is waterproof and airtight.

Any of the aspects herein, wherein a surface of the finger is in contact with a side surface of the robot mount bracket, and wherein the contact prevents rotation of the clamp relative to the robot mount bracket as the clamp is moved between the clamped state and the unclamped state.

Any of the aspects herein, wherein the rotational attachment comprises a hole disposed through the robot mount bracket with a counterbore disposed on the robot attachment surface, wherein a shoulder bolt is disposed in the hole and is fastened to the clamp screw, and wherein a head of the shoulder bolt is disposed in the counterbore.

Any of the aspects herein, wherein the hole comprises an axis that runs parallel to the longitudinal axis of the elongate arm, and wherein the direction away from the longitudinal axis of the elongate arm is perpendicular to the longitudinal axis.

Any of the aspects herein, wherein the hook portion is disposed closer to the robot attachment surface when in the clamped state than when in the unclamped state.

Any of the aspects herein, wherein the hook portion joins the first arm to the second arm.

Any of the aspects herein, wherein the hook portion comprises a first hook portion disposed on an end of the first arm and a second hook portion disposed on the end of the second arm, and wherein the first hook portion and the second hook portion are separate from one another.

A robotic system according to at least one embodiment of the present disclosure comprises: a robot arm comprising a plurality of links and joints running from a distal end to a proximal end; an end-effector foot attached to the robot arm at the proximal end; and a registration assembly attached to the end-effector foot, the registration assembly comprising: an elongate arm extending longitudinally from a proximal arm end to a distal arm end, the elongate arm comprising: a target receiving surface disposed at the distal arm end; and a robot mount bracket disposed at the proximal arm end and protruding from the elongate arm in a direction away from a longitudinal axis of the elongate arm, the robot mount bracket comprising a robot attachment surface disposed at a proximal end of the robot mount bracket; a clamp screw extending a length along a rotation axis from a first end to a second end, the clamp screw comprising: a bracket contact surface disposed at the first end, wherein the bracket contact surface is in contact with the robot mount bracket; and a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis; and a clamp, comprising: a plate; a threaded hole running through the plate, wherein the threaded outer surface of the clamp screw is threadedly engaged with the threaded hole; and a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp.

Any of the aspects herein, wherein the clamp screw comprises a thumb wheel disposed at the second end.

Any of the aspects herein, wherein the registration assembly is attached to the end-effector foot in the clamped state, and wherein the registration assembly is releasable from being attached to the end-effector foot in the unclamped state.

Any of the aspects herein, wherein the registration assembly further comprises: a registration target frame mounted to the target receiving surface of the elongate arm, the registration target frame comprising marker beads that are arranged in a pattern on at least one surface of the registration target frame.

Any of the aspects herein, wherein the registration assembly further comprises: a sterile sleeve sealed around and fully enclosing the registration assembly maintaining an environment inside the sterile sleeve from an environment outside the sterile sleeve.

Any of the aspects herein, wherein the registration assembly is attached to the end-effector foot in the clamped state without penetrating the sterile sleeve.

Any of the aspects herein, wherein the registration assembly is separable from the end-effector foot, in the unclamped state, while maintaining the environment inside the sterile sleeve from the environment outside the sterile sleeve.

Any of the aspects herein, wherein a robot sterile sleeve covers the end-effector foot, and wherein an unpenetrated portion of the robot sterile sleeve and an unpenetrated portion of the sterile sleeve are clamped between the robot mount bracket and the end-effector foot along the robot attachment surface.

A sealed robotic registration assembly according to at least one embodiment of the present disclosure comprises: an elongate arm extending longitudinally from a proximal end to a distal end, the elongate arm comprising: a target receiving surface disposed at the distal end; and a robot mount bracket disposed at the proximal end and protruding from the elongate arm in a direction away from a longitudinal axis of the elongate arm, the robot mount bracket comprising a robot attachment surface disposed at a proximal end of the robot mount bracket; a registration target frame mounted to the target receiving surface of the elongate arm, the registration target frame comprising marker beads arranged in a pattern on at least one surface of the registration target frame; a clamp screw extending a length along a rotation axis from a first end of the clamp screw to a second end of the clamp screw, the clamp screw comprising: a bracket contact surface disposed at the first end, wherein the bracket contact surface is in contact with the robot mount bracket; and a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis; a clamp, comprising: a plate; a threaded hole running through the plate, wherein the threaded outer surface of the clamp screw is threadedly engaged with the threaded hole; and a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp; and a sterile sleeve sealed around and fully enclosing the elongate arm, the registration target frame, the clamp screw, and the clamp maintaining an environment inside the sterile sleeve from an environment outside the sterile sleeve.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a perspective diagram of a robotic surgical system robot according to at least one embodiment of the present disclosure;

FIG. 3A is a detail perspective view of a robotic registration assembly attached to the robotic surgical system of FIG. 2 according to at least one embodiment of the present disclosure;

FIG. 3B is a detail section view of a sealed mount interface between the robotic registration assembly and the robotic surgical system of FIG. 3A;

FIG. 3C is a perspective view of a clamp of the robotic registration assembly shown in FIG. 3A;

FIG. 3D is a detail perspective view of an end-effector foot of the robotic surgical system according to at least one embodiment of the present disclosure;

FIG. 4A is a detail perspective view of a robotic registration assembly attached to the robotic surgical system of FIG. 2 according to at least one embodiment of the present disclosure;

FIG. 4B is a detail section view of a sealed mount interface between the robotic registration assembly and the robotic surgical system of FIG. 4A;

FIG. 4C is a perspective view of a clamp of the robotic registration assembly shown in FIG. 3A:

FIG. 5A is a perspective view of a robotic registration assembly in a sterile sleeve comprising a first registration target frame according to at least one embodiment of the present disclosure; and

FIG. 5B is a perspective view of a robotic registration assembly in a sterile sleeve comprising a second registration target frame according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple All, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some examples, the present disclosure relates generally to a reusable robotic registration kit and, more specifically, to an image registration marker (3D marker or Star marker) reusable kit for spinal robotics image registration needed between pre-operated CT image and intra-operated C-arm or O-arm image. Typically, spinal robotics image registration needs pre-operated CT images and intra-operated C-arm and O-arm images with specific 3D marker beads pattern for C-arm image and Star marker beads pattern for O-arm image. The beads pattern in the 3D marker and Star marker requires precise positional accuracy. During C-arm and O-arm image taking, the 3D marker and Star marker are assembled to the robotics arm end and placed just above the region of interest of the patient. Both the registration target frame (e.g., the 3D marker, Star marker, etc.) and the robotics arm have a sterilization requirement (e.g., the components must remain sterile in a surgical environment. After the image registration, the 3D marker/Star marker must be removed from the robotics arm end (e.g., end-effector foot) and then the surgical procedure can be performed. During the attachment and removal of the registration target frame, and while performing the surgical procedure, the robotic arm must remain sterile.

In conventional systems, the 3D marker (e.g., for a C-arm image registration) and Star marker (e.g., for an O-arm registration) are designed for disposable use. Each procedure requires one set of brand new 3D marker or Star marker. As can be appreciated, disposing of these components after a single use results in a high procedure cost. This cost can be significant, especially in emerging markets. Moreover, disposing the components after a single use results in the accumulation of medical waste.

Conventional registration kits generally include a registration target frame, or marker (e.g., 3D marker, Star marker, etc.), a target extender, an arm guide, and a screw that fastens the arm guide to the robotic arm end. The target extender, arm guide, and screw may be reusable auto-clave kits. After assembling the arm guide into the robotic arm end, which is covered by a sterile drape, the 3D marker/Star marker is mounted into the target extender parts. The arm guide is attached to the robotic arm end by the screw, which penetrates the sterile drape. After the registration phase is completed, the 3D marker/Star marker would be scrapped (e.g., disposed of) and the target extender and screw are kept for cleaning (e.g., by autoclaving, etc.) before the next procedure. The arm guide remains assembled to the robotic arm end for performing the require procedural operations (e.g., surgical operations, etc.).

It is with respect to the above issues and other problems that the embodiments presented herein were contemplated.

In one example, the registration target frame, the target extender, the arm guide, and the mount screw are combined as an integrated kit that is capable of being completely covered by a sterile sleeve, or drape, during image registration. This reusable registration assembly may attach to the robotic arm end via a clamp interface, preventing the sterile sleeve from being penetrated by a screw as in conventional approaches. The clamp may be moved between a clamped state and an unclamped state by rotating a clamp screw that is disposed inside the sterile sleeve. The clamp screw may include a thumb wheel, or handle, that can be gripped from outside of the sterile sleeve and rotated in repeated quarter turns, or the like, without damaging the sterile sleeve or penetrating the sterile environment inside the sterile sleeve.

The clamp may be in any shape that allows a portion of the sterile sleeve to be disposed between and around the clamping surfaces and the robotic arm end. In one example, the clamp may have one or more fingers that contact the robotic arm end. For example, the finger may comprise a hook portion that engages with a receiving feature on the robotic arm end. In one example, the clamp may comprise multiple separate fingers, each with a corresponding hook portion. In another example, the clamp may comprise fingers that are joined together by a spanning hook portion. The spanning hook portion may allow the reusable registration assembly to be quickly installed to the robotic arm end, especially in cases where multiple image registrations are needed during a procedure, or series of procedures. In any event, the reusable registration assembly can attach to the robotic arm end without any parts protruding from the sterile sleeve, and without penetrating the sterile sleeve.

Embodiments of the present disclosure provide technical solutions to one or more of the problems of (1) disposing of the registration target frame after each use, creating waste, (2) only allowing one use of a registration target frame before disposal, (3) a cumbersome, slow, and costly assembly of components each time a registration is required, (4) not maintaining any of the components in a sterile environment during use, (5) requiring cleaning of multiple parts (not the registration target frame, which is scrapped) after each use, etc. In one example, the technical solutions may include providing a reusable registration assembly that can be contained in a sterile sleeve. The reusable registration assembly may be attached to and/or removed from a robotic arm end without compromising the sterility of the reusable registration assembly. For instance, the reusable registration assembly can mount to the robotic arm end without penetrating the sterile sleeve with a screw, pin, or hole. By keeping the reusable registration assembly inside a sterile sleeve, the reusable registration assembly, including the registration target frame (e.g., marker), can be removed from the robotic arm end after use, taken to a cleaning station, where the sterile sleeve can be wiped down or otherwise cleaned, and then the reusable registration assembly can be removed from the sterile sleeve and inserted into, and sealed inside, a new sterile sleeve for subsequent use. As can be appreciated, at least some benefits of the reusable registration assembly described herein include reduced procedural costs and reduced waste. Moreover, the reusable registration assembly may be configured as a kit that is contained within a sterile sleeve ready to be mounted to a robotic arm end. Among other things, this reusable registration assembly, or kit, allows for faster mounting (e.g., attachment), faster demounting (e.g., detachment), and faster changeover between registration operations and surgical operations than conventional systems. This sterile and quick-install approach allows for shorter overall surgical times as well as increased accuracy of movement for the robot since the time from calibration (e.g., via the registration target frame, etc.) to operation is shorter. The overall surgical times are shorter at least because the required effort, or amount of time, to assemble the reusable registration assembly to the robotic arm end is shorter than conventional multi-component non-sterile kits. The accuracy of movement for the robot is increased at least since the time between calibration (with the reusable registration assembly) and operation (e.g., surgical operation, etc., by the robot) is shorter than conventional times preventing any drift, fluctuation, or deterioration in registration for the robot.

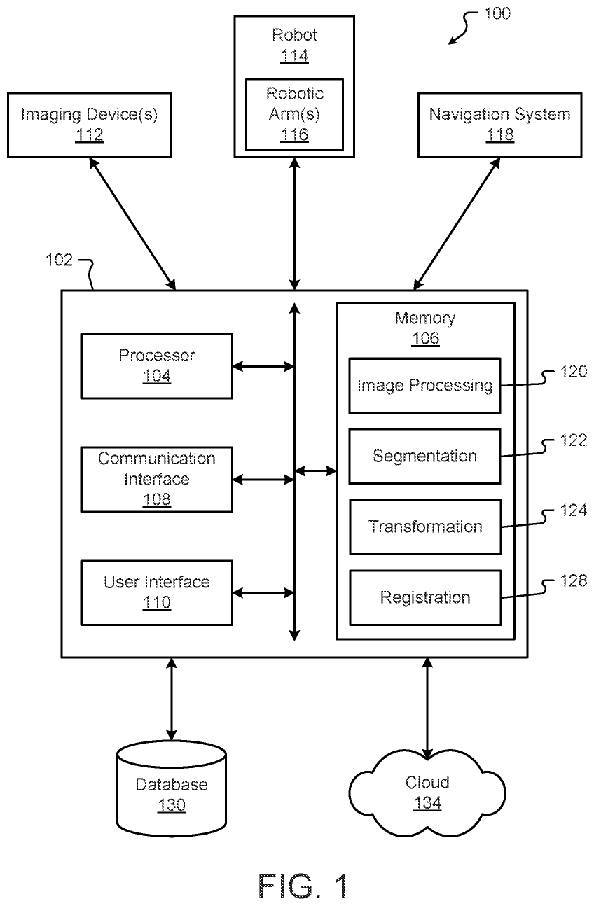

Turning first to FIG. 1, a block diagram of a system 100 according to at least one embodiment of the present disclosure is shown. The system 100 may be used to operate a robot 114 according to one of several defined movement paths or kinematic solutions associated with, and specific to, a connected end effector. In some examples, the system 100 may control, pose, and/or otherwise manipulate a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X1 Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

In some examples, the imaging device 112 and the navigation system 118 may determine a spatial registration of the robot 114 based on registration with a reusable registration assembly. For instance, the reusable registration assembly may comprise a registration target frame comprising marker beads that are arranged in a pattern on at least one surface of the registration target frame. These marker beads may be monitored by the imaging device 112 as the robot 114 is actuated or positioned in various poses to determine a spatial registration, and/or relative movement kinematics, for the robotic arm 116. In some examples, this registration may be referred to herein as “spatial registration,” “robotic movement calibration,” or “calibration.”

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. In one example, the database 130 may comprise movement profiles for the robot 114 based on a select end effector that is attached to the robotic arm 116. These movement profiles may correspond to kinematic solutions for the robot 114 and/or defined positions of a surgical tool axis of select end effector relative to at least one of a surface of a tool block of the end effector and a rotation axis of a final joint/mount flange of the robotic arm 116. In some examples, the database 130 may store identifications of specific tool blocks and surgical tool axis orientations. In any event, the database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods described herein. The system 100 or similar systems may also be used for other purposes.

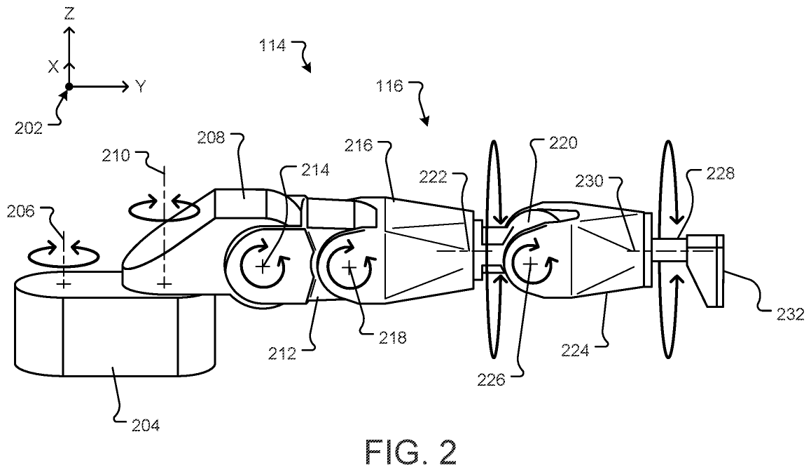

Referring now to FIG. 2, a perspective diagram of a robotic surgical system robot 114 is shown in accordance with examples of the present disclosure. More specifically, FIG. 2 shows the robotic arm 116 of the robot 114 and an end-effector foot 232 to which a surgical tool and/or a registration assembly may be attached.

Features of the robot 114 and/or robotic arm 116 may be described in conjunction with a coordinate system 202. The coordinate system 202, as shown in FIG. 2, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 202 may be placed at any point on or near the components of the robot 114, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robot 114 and/or robotic arm 116 with respect to the coordinate system 202. For example, the width of the robotic arm 116 (e.g., running from the side shown in the foreground to the side in the background, into the page) may be defined as a dimension along the X-axis of the coordinate system 202, the height of the robotic arm 116 may be defined as a dimension along the Z-axis of the coordinate system 202, and the length of the robotic arm 116 (e.g., running from a proximal end at the first link 204 to a distal end at the seventh link 224, etc.) may be defined as a dimension along the Y-axis of the coordinate system 202. Additionally or alternatively, the height of the system 100 may be defined as a dimension along the Z-axis of the coordinate system 202, a reach of the robotic arm 116 may be defined as a dimension along the Y-axis of the coordinate system 202, and a working area of the robotic arm 116 may be defined in the XY-plane with reference to the corresponding axes of the coordinate system 202.

The robotic arm 116 may be comprised of a number of links 204, 208, 212, 216, 220, 224, 228 that interconnect with one another at respective axes of rotation 206, 210, 214, 218, 222, 226, 230, or joints. There may be more or fewer links 204, 208, 212, 216, 220, 224, 228 and/or axes of rotation 206, 210, 214, 218, 222, 226, 230 than are shown in FIG. 2. In one example, the robot 114 and/or the robotic arm 116 may be shrouded, draped, or sealed within, a robot sterile sleeve. In any event, the robotic arm 116 may have a first link 204 disposed at a proximal end of the robotic arm 116 and an end-effector foot 232 disposed furthest from the proximal end at a distal end of the robotic arm 116. The first link 204 may correspond to a base of the robotic arm 116. In some examples, the first link 204 may rotate about first rotation axis 206. A second link 208 may be connected to the first link 204 at a second rotation axis 210, or joint. The second link 208 may rotate about the second rotation axis 210. In one example, the first rotation axis 206 and the second rotation axis 210 may be arranged parallel to one another. For instance, the first rotation axis 206 and the second rotation axis 210 are shown extending along the Z-axis in a direction perpendicular to the XY-plane.

The robotic arm 116 may comprise a third link 212 that is rotationally interconnected to the second link 208 via the third rotation axis 214, or joint. The third rotation axis 214 is shown extending along the X-axis, or perpendicular to the first rotation axis 206 and second rotation axis 210. In this position, when the third link 212 is caused to move (e.g., rotate relative to the second link 208), the third link 212 (and the components of the robotic arm 116 extending from the third link 212) may be caused to move into or out of the XY-plane. The fourth link 216 is shown rotationally interconnected to the third link 212 via the fourth rotation axis 218, or joint. The fourth rotation axis 218 is arranged parallel to the third rotation axis 214. The fourth rotation axis 218 extends along the X-axis allowing rotation of the fourth link 216 into and out of the XY-plane.

In some examples, the robotic arm 116 may comprise one or more wrists 220, 228. The fifth link 220, or wrist, is shown rotationally interconnected to the fourth link 216 via a fifth rotation axis 222, or wrist joint. The fifth rotation axis 222 is shown extending along the Y-axis, which is perpendicular to the X-axis and the Z-axis. During operation of the robot 114, causing the fifth link 220 to rotate about the fifth rotation axis 222 may cause the components of the robotic arm 116 distal the joint at the fifth rotation axis 222 (e.g., the fifth link 220, the sixth link 224, the seventh link 228, the end-effector foot 232, etc.) to rotate about the Y-axis.

The sixth link 224 is rotationally interconnected to the fifth link 220 via the sixth rotation axis 226. The sixth rotation axis 226 extends along the X-axis and provides for rotation of the sixth link 224 relative to the fifth link 216 (e.g., into and out of the XY-plane in the position shown).

The seventh link 228, or wrist, is shown rotationally interconnected to the sixth link 224 via a seventh rotation axis 230, or wrist joint. The seventh rotation axis 230 is shown extending along the Y-axis (e.g., perpendicular to the X-axis and the Z-axis). During operation of the robot 114, causing the seventh link 228 to rotate about the seventh rotation axis 230 may cause the components of the robotic arm 116 distal the joint at the seventh rotation axis 230 (e.g., the end-effector foot 232, an attached registration assembly, and/or surgical tool, etc.) to rotate about the Y-axis. In some examples, the seventh link 228 may move axially along the seventh rotation axis 230. Causing the seventh link 228 to move axially along the seventh rotation axis 230 may cause the components of the robotic arm 116 distal the joint at the seventh rotation axis 230 (e.g., the end-effector foot 232, an attached registration assembly, and/or surgical tool, etc.) to translate axially (e.g., linearly) along the Y-axis.

Located at the distal end of the robotic arm 116, an end-effector foot 232 may be interconnected to the seventh link 228. In some examples, the end-effector foot 232 may include one or more mount interfaces to surgical tools, registration assemblies, and/or the like. The end-effector foot 232 may be affixed to the seventh link 228 such that movement of the seventh link 228 (e.g., rotation and/or translation) results in a corresponding movement of the end-effector foot 232.

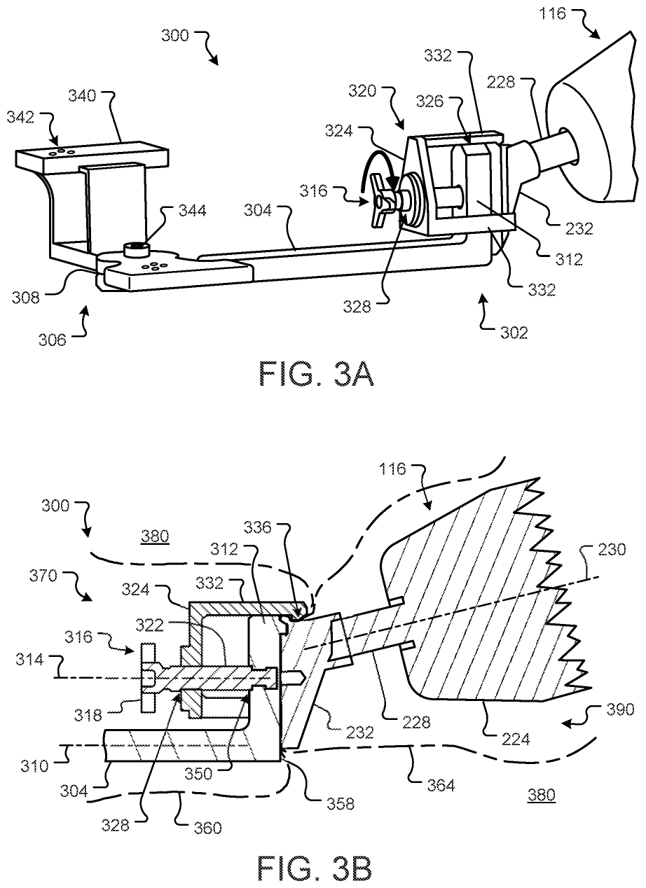

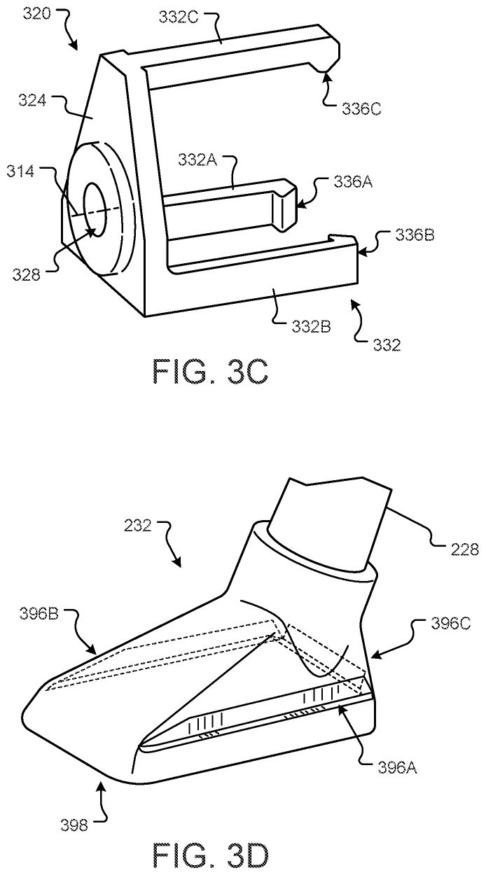

FIGS. 3A and 3B show views of a registration assembly 300 (e.g., reusable registration kit, etc.) attached to the end-effector foot 232 of the robotic arm 116 in a clamped state in accordance with examples of the present disclosure. The registration assembly 300 may include an elongate arm 304, a clamp screw 316, a clamp 320, and a registration target frame 340. While embodiments of the present disclosure describe that the registration assembly 300 may be attached to the robotic arm 116 while contained in a sterile sleeve, the sterile sleeve is not shown in FIG. 3A for the sake of clarity in disclosure. The terms “robotic registration kit,” “registration kit,” “reusable registration kit,” “robotic registration assembly,” “registration assembly,” “reusable registration assembly,” “sealed robotic registration assembly,” and/or variations thereof may be used interchangeably herein.

The elongate arm 304 may extend longitudinally from a proximal end 302 (e.g., a proximal arm end) to a distal end 306 (e.g., a distal arm end) thereof. The elongate arm 304 may be a rigid member made from metal (e.g., stainless steel, aluminum, titanium, and/or the like), plastic (e.g., thermoplastic, polyethylene terephthalate, high-density polyethylene, polyvinyl chloride, polypropylene, and/or the like), and/or combinations thereof. At the proximal end 302, a robot mount bracket 312 may be formed from the elongate arm 304 and extend, or protrude, in a direction away from a longitudinal axis 310 of the elongate arm. In some examples, the direction away from the longitudinal axis 310 may be disposed perpendicular, or at approximately 90 degrees (plus or minus 5 degrees), to the longitudinal axis 310. The robot mount bracket 312 may comprise a robot attachment surface 358 that is caused to clamp against the end-effector foot 232, when the registration assembly 300 is in the clamped state. At the distal end 306, a target receiving surface 308 is disposed on a portion of the elongate arm 304. The target receiving surface 308 may correspond to a recess or cutout that is configured to receive a registration target frame 340. In one example, the registration target frame 340 may be attached to the target receiving surface 308 via at least one marker mount screw 344. The registration target frame 340 may correspond to any marker or reference frame comprising marker beads 342 arranged in a pattern on at least one surface thereof. The registration target frame 340 may be configured as a 3D marker, a Star marker, or any other marker having measurement fiducials, reference marks, and positional indicia that are capable of being interpreted (e.g., by the imaging devices 112, the navigation system 118, etc.) by the system 100 in determining a spatial registration for the robot 114 and/or robotic arm 116.

A clamp screw 316 may be rotationally attached to the robot mount bracket 312 of the elongate arm 304 at a rotational attachment 350. The rotational attachment 350 may correspond to a hole comprising one or more counterbores. In one example, the clamp screw 316 may, at a first end, include a bracket contact surface that contacts the robot mount bracket 312. The bracket contact surface may be disposed in a counterbore of the robot mount bracket 312. A shoulder bolt, or pin, may be inserted into the first end of the clamp screw 316 from the robot attachment surface 358 side of the robot mount bracket 312. The clamp screw 316 may be allowed to rotate about the rotation axis 314 but may be restricted from moving axially via the rotational attachment 350. For example, the shoulder bolt may prevent the clamp screw 316 from being removed in a direction opposite the end-effector foot 232 and/or the robot mount bracket 312. Continuing this example, the bracket contact surface may prevent the clamp screw 316 from moving axially in a direction toward the end-effector foot 232 and/or the robot mount bracket 312. The clamp screw 316 may comprise a threaded outer surface 322 that threadedly engages with a threaded hole 328 of the clamp 320. As the clamp screw 316 is rotated about the rotation axis 314 (e.g., by turning the clamp screw 316 at the thumb wheel 318, etc.), the clamp 320 is caused to move axially (e.g., in an axial direction along the rotation axis 314) between a clamped state and an unclamped state. When a right-handed thread is utilized for the threaded outer surface 322 and the threaded hole 328, a clockwise turn of the thumb wheel 318 (as shown by the rotation arrow in FIG. 3A) causes the clamp 320 to move toward the thumb wheel 318 (e.g., such that the plate 324 of the clamp 320 is disposed closer to the thumb wheel 318 at the second end of the clamp screw 316). As the clamp 320 moves in this direction, the hook portion 336 of the one or more fingers 332 may engage with corresponding grooves in the end-effector foot 232 clamping the registration assembly 300 to the end-effector foot 232 and robotic arm 116. The clamp screw 316 may be made from metal (e.g., stainless steel, aluminum, titanium, and/or the like), plastic (e.g., thermoplastic, polyethylene terephthalate, high-density polyethylene, polyvinyl chloride, polypropylene, and/or the like), and/or combinations thereof.

The registration assembly 300 may comprise a sterile sleeve 360 that fully encloses the registration assembly 300 (e.g., the robotic registration kit) maintaining a sterile environment 370 (inside the sterile sleeve 360) from the surgical environment 380 (e.g., an environment outside the sterile sleeve 360). Although the surgical environment 380 may also be considered a sterile environment, the sterile environment 370 inside the sterile sleeve 360 may need to be kept separate from the surgical environment 380 to avoid contamination of the registration assembly 300 by elements in the surgical environment 380 (e.g., blood, bodily fluids, skin, gasses, and/or other elements). In this manner, the registration assembly 300 may be later removed from the sterile sleeve 360 and reused without requiring cleaning and/or sterilization of the registration assembly 300 (as would be required if the registration assembly 300 was not maintained in the sterile environment 370 of the sterile sleeve 360). The sterile sleeve 360 may comprise a thin film bag sealed around the registration assembly 300. The sterile sleeve 360 is waterproof and airtight. In one example, the sterile sleeve 360 may be a transparent or clear plastic material. As illustrated in FIG. 3B, the registration assembly 300 may be clamped to the end-effector foot 232 while the registration assembly 300 is fully enclosed in, and/or sealed inside, the sterile sleeve 360. In FIG. 3B, the robotic arm 116 may be draped in a robot sterile sleeve 364. It is an aspect of the present disclosure that the registration assembly 300 is clamped to the end-effector foot 232 without penetrating any portion of the sterile sleeve 360 and/or the robot sterile sleeve 364. In contrast to a mount that would require a screw to pass through the sterile sleeve 360 and/or the robot sterile sleeve 364 (e.g., compromising at least one sterile environment 370, 390 of the system 100 by allowing the registration assembly 300 to be exposed to the surgical environment 380, etc.), the present disclosure describes a clamp mount that maintains the sterility of the registration assembly 300 within the unpenetrated sterile sleeve 360. In some examples, and as shown in FIG. 3B, an unpenetrated portion of the robot sterile sleeve 364 and an unpenetrated portion of the sterile sleeve 360 are clamped between the robot mount bracket 312 and the end-effector foot 232 along the robot attachment surface 358.

In one example, the rotation axis 314 of the clamp screw 316 may be arranged parallel to the longitudinal axis 310 of the elongate arm 304. As the clamp 320 is moved between a clamped state and an unclamped state (e.g., by rotating the clamp screw 316), an anti-rotation contact surface 326 of the finger 332 may slide along a side surface of the robot mount bracket 312. This contact may prevent rotation of the clamp 320 (e.g., about the rotation axis 314) as the clamp screw 316 is rotated. Stated another way, the anti-rotation contact surface 326 in contact with the side surface of the robot mount bracket 312 may cause the rotation of the clamp screw 316 to result in a linear movement of the clamp 320 in an axial direction parallel to the rotation axis 314 and/or the longitudinal axis 310.

FIG. 3C is a perspective view of a clamp 320 of the registration assembly 300. The clamp 320 comprises a plate 324, a threaded hole 328 running through the plate 324, and at least one finger 332 formed from the plate 324 and extending a distance to a clamp end of the finger 332. When included as part of the registration assembly 300, the clamp end may comprise a hook portion 336 that is disposed offset proximally past the robot attachment surface 358 of the robot mount bracket 312. As shown in FIG. 3C, the finger 332 comprises a first arm 332A, a second arm 332B, and a third arm 332C. Each of the arms 332A-332C include a corresponding hook portion 336A-336C. For instance, the first arm 332A comprises a first hook portion 336A, the second arm 332B comprises a second hook portion 336B, and the third arm 332C comprises a third hook portion 336C. The first arm 332A is shown disposed on a first side (e.g., corresponding to a first side of the elongate arm 304) and the second arm 332B is shown disposed on a second side (e.g., corresponding to a second side of the elongate arm 304 that is arranged opposite the first side of the elongate arm 304). The third arm 332C is shown disposed between the first arm 332A and the second arm 332B. In some examples, the clamp 320 may be symmetrical about a center plane running through the third arm 332C and the rotation axis 314. The clamp 320 may be made from metal (e.g., stainless steel, aluminum, titanium, and/or the like), plastic (e.g., thermoplastic, polyethylene terephthalate, high-density polyethylene, polyvinyl chloride, polypropylene, and/or the like), and/or combinations thereof.

In some examples, the end-effector foot 232 may comprise one or more features that engage with or receive a portion of the clamp 320. FIG. 3D shows a detail perspective view of an end-effector foot 232 of the robotic arm 116 according to at least one embodiment of the present disclosure. The end-effector foot 232 may comprise one or more clamp grooves 396A-396C. These clamp grooves 396A-396C may correspond to a cutout, relief, ledge, milled recess, or other interrupted surface that mates with features of the clamp 320. For instance, the clamp grooves 396A-396C may mate, or otherwise engage, with respective hook portions 336A-336C of the clamp 320. More specifically, the first hook portion 336A may engage with the first clamp groove 396A, the second hook portion 336B may engage with the second clamp groove 396B, and the third hook portion 336C may engage with the third clamp groove 396C. The shape of each of the clamp grooves 396A-396C may match, or mirror, a shape of the hook portions 336A-336C. As the clamp 320 is tightened (e.g., by turning the clamp screw 316 in a clockwise direction as described above) and the robot attachment surface 358 is brought closer to the registration assembly mount surface 398 of the end-effector foot 232, the hook portions 336A-336C may seat against the clamp grooves 396A-396C. Although shown as including a plurality of clamp grooves 396A-396C, the hook portions 336A-336C may engage with a shape of the end-effector foot 232 absent these clamp grooves 396A-396C. For instance, the hook portions 336A-336C may wrap partially, or even completely, around a portion of the body of the end-effector foot 232.

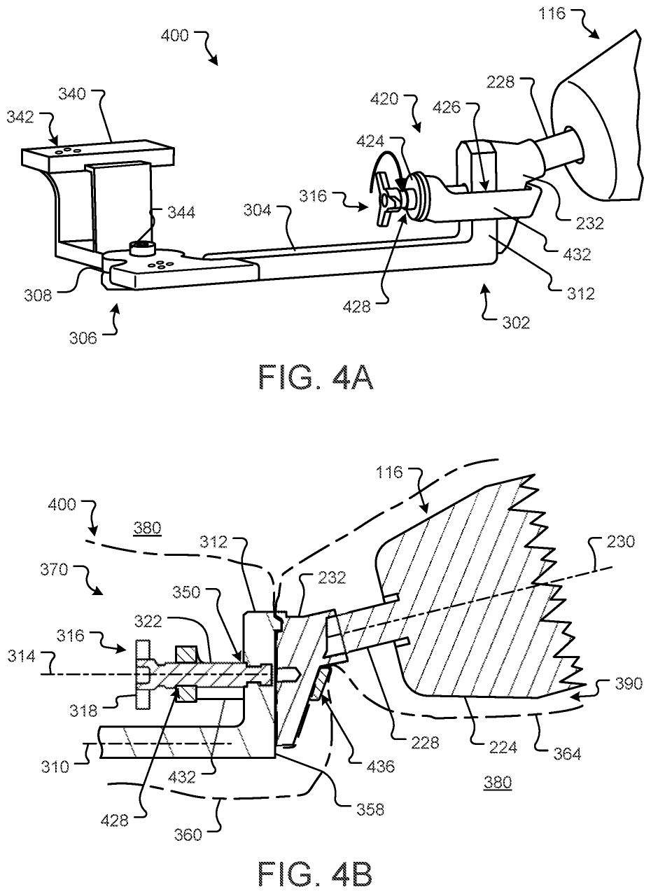

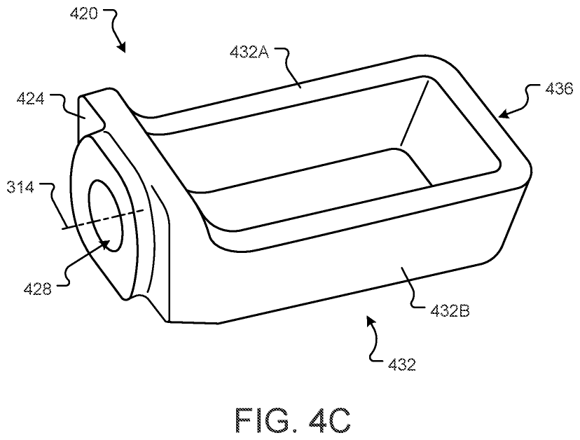

Referring now to FIGS. 4A and 4B, various views of a registration assembly 400 (e.g., reusable registration kit, etc.) are shown attached to the end-effector foot 232 of the robotic arm 116 in a clamped state in accordance with examples of the present disclosure. The registration assembly 400 may comprise similar, if not identical, components as described in conjunction with FIGS. 3A and 3B. For instance, the registration assembly 400 may include an elongate arm 304, a clamp screw 316, a clamp 320, and a registration target frame 340. Any use of a reference character for a component described in conjunction with FIGS. 4A-4C that matches a reference character for a component described in conjunction with FIGS. 1-3D indicates a same element or a common element. Accordingly, duplicate description of these components may be omitted for the sake of brevity and clarity in disclosure. While embodiments of the present disclosure describe that the registration assembly 400 may be attached to the robotic arm 116 while contained in a sterile sleeve, the sterile sleeve is not shown in FIG. 4A for the sake of clarity in disclosure.

In some examples, the clamp 320 of FIGS. 3A-3C may be replaced by the closed end clamp 420 shown in FIGS. 4A-4C. Similar to FIGS. 3A-3C, the clamp screw 316 may comprise a threaded outer surface 322 that threadedly engages with a threaded hole 428 of the clamp 420. As the clamp screw 316 is rotated about the rotation axis 314 (e.g., by turning the clamp screw 316 at the thumb wheel 318, etc.), the clamp 420 is caused to move axially (e.g., in an axial direction along the rotation axis 314) between a clamped state and an unclamped state. When a right-handed thread is utilized for the threaded outer surface 322 and the threaded hole 428, a clockwise turn of the thumb wheel 318 (as shown by the rotation arrow in FIG. 4A) causes the clamp 420 to move toward the thumb wheel 318 (e.g., such that the plate 424 of the clamp 420 is disposed closer to the thumb wheel 318 at the second end of the clamp screw 316). As the clamp 420 moves in this direction, the hook portion 436 of the finger 432 may engage with a surface of the end-effector foot 232 clamping the registration assembly 400 to the end-effector foot 232 and the robotic arm 116.

The registration assembly 400 may comprise a sterile sleeve 360 that fully encloses the registration assembly 400 maintaining a sterile environment 370 (inside the sterile sleeve 360) from the surgical environment 380 (e.g., an environment outside the sterile sleeve 360). The sterile sleeve 360 may comprise a thin film (e.g., clear, semi-transparent, or transparent plastic, etc.) bag sealed around the registration assembly 400. This sterile sleeve 360 may be waterproof and airtight. As illustrated in FIG. 4B, the registration assembly 400 may be clamped to the end-effector foot 232 while the registration assembly 400 is fully enclosed in, and/or sealed inside, the sterile sleeve 360. In FIG. 4B, the robotic arm 116 is shown draped in a robot sterile sleeve 364. It is an aspect of the present disclosure that the registration assembly 400 is clamped to the end-effector foot 232 without penetrating any portion of the sterile sleeve 360 and/or the robot sterile sleeve 364. In contrast to a mount that would require a screw to pass through the sterile sleeve 360 and/or the robot sterile sleeve 364 (e.g., compromising at least one sterile environment 370, 390 of the system 100 by allowing the registration assembly 400 to be exposed to the surgical environment 380, etc.), the present disclosure describes a clamp mount that maintains the sterility of the registration assembly 400 within the unpenetrated sterile sleeve 360. For example, the end-effector foot 232, covered in at least a portion of the robot sterile sleeve 364, may be inserted into a space of the closed end clamp 420 between the fingers 432 and hook portion 436. Continuing this example, as the end-effector foot 232 enters the space, a portion of the sterile sleeve 360 of the registration assembly 400 may displace inside the space, without tearing or being penetrated. Once inserted, the clamp screw 316 may be rotated about the rotation axis 314 and the finger 432 may pull the end-effector foot 232 against the robot attachment surface 358 until the registration assembly 400 is in a clamped state. In some examples, and as shown in FIG. 4B, an unpenetrated portion of the robot sterile sleeve 364 and an unpenetrated portion of the sterile sleeve 360 are clamped between the robot mount bracket 312 and the end-effector foot 232 along the robot attachment surface 358.

When the clamp 420 is moved between a clamped state and an unclamped state (e.g., by rotating the clamp screw 316), an anti-rotation contact surface 426 of the finger 432 may slide along a side surface of the robot mount bracket 312. This contact may prevent rotation of the clamp 420 (e.g., about the rotation axis 314) as the clamp screw 316 is rotated. Stated another way, the anti-rotation contact surface 426 in contact with the side surface of the robot mount bracket 312 may cause the rotation of the clamp screw 316 to result in a linear movement of the clamp 420 in an axial direction parallel to the rotation axis 314 and/or the longitudinal axis 310.

FIG. 4C shows a perspective view of a clamp 420 of the registration assembly 400 in accordance with examples of the present disclosure. The clamp 420 comprises a plate 424, a threaded hole 428 running through the plate 424, and at least one finger 432 formed from the plate 424 and extending a distance to a clamp end of the finger 432. When included as part of the registration assembly 400, the clamp end may comprise a hook portion 436 that is disposed offset proximally past the robot attachment surface 358 of the robot mount bracket 312. As shown in FIG. 4C, the finger 432 comprises a first arm 432A and a second arm 432B. The first arm 432A may be joined to the second arm 432B by the hook portion 436. For example, the hook portion 436 may span from the first arm 432A to the second arm 432B. The first arm 432A is shown disposed on a first side (e.g., corresponding to a first side of the elongate arm 304) and the second arm 432B is shown disposed on a second side (e.g., corresponding to a second side of the elongate arm 304 that is arranged opposite the first side of the elongate arm 304). The hook portion 436 may comprise at least one surface that matches, or mirrors, a surface of the end-effector foot 232. In some examples, the clamp 320 may be symmetrical about a center plane that is parallel the first arm 432A and the second arm 432B and that runs through the rotation axis 314. The clamp 420 may be made from metal (e.g., stainless steel, aluminum, titanium, and/or the like), plastic (e.g., thermoplastic, polyethylene terephthalate, high-density polyethylene, polyvinyl chloride, polypropylene, and/or the like), and/or combinations thereof.

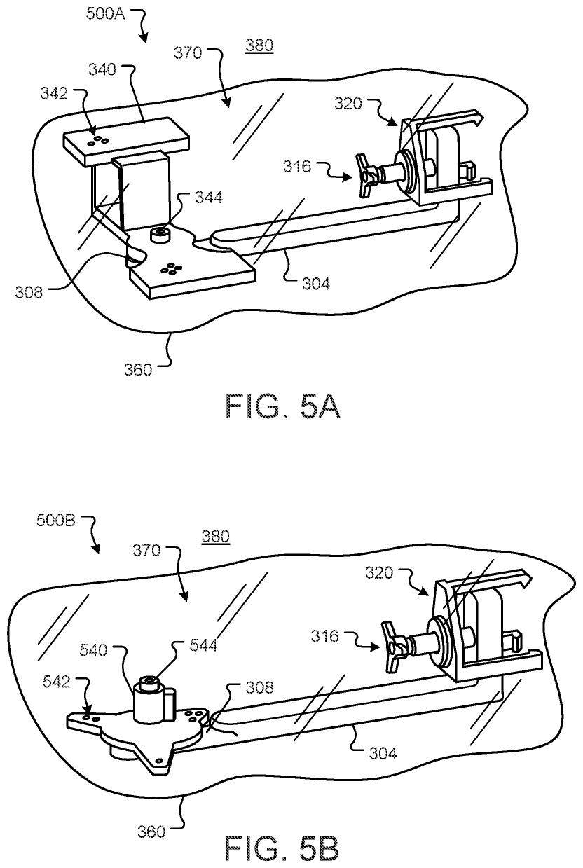

FIGS. 5A and 5B show perspective views of robotic registration assemblies 500A, 500B sealed inside a sterile sleeve 360 and comprising first and second registration target frames 340, 540, respectively. The first and second registration assemblies 500A, 500B may correspond to a reusable registration kit that is capable of being selectively attached to, and removed from, a robotic arm 116 without puncturing, penetrating, tearing, or otherwise compromising the sterile sleeve 360. In this manner, the sterility of the registration assembly 300, 400 contained therein is maintained.

As illustrated in FIGS. 5A and 5B, the clamp 320 of the first registration assembly 500A is shown in an unclamped state. In the unclamped state, the hook portion 336 of the clamp 320 is disposed further from the robot attachment surface 358 than when in the clamped state. Additionally or alternatively, the plate 324 of the clamp 320 is disposed further from the thumb wheel 318 in the unclamped state than when in the clamped state, or vice versa. When attached to a robotic arm 116 in the clamped state, the registration assembly 300, 400, contained inside the sterile sleeve 360, may be affixed to the robotic arm 116 and may move with the robotic arm 116. In the clamped state, the registration assembly 300, 400 may be maintained in the affixed position until the clamp screw 316 is unscrewed and the clamp 320, 420 is moved into the unclamped state or position.

The first registration assembly 500A, shown in FIG. 5A, may correspond to the registration assembly 300 (e.g., described in conjunction with FIGS. 3A-3D, etc.) or the registration assembly 400 (e.g., described in conjunction with FIGS. 4A-4C, etc.) with a registration target frame 340 mounted to the target receiving surface 308 of the elongate arm 304. The registration target frame 340 may correspond to a 3D marker that includes one or more surfaces disposed at different heights and/or distances from the target receiving surface 308. The registration target frame 340 may include marker beads 342 or other fiducials that are arranged in a pattern on the one or more surfaces of the registration target frame 340. The registration target frame 340 may be removably attached to the target receiving surface 308 via at least one marker mount screw 344. In one example, the registration target frame 340 may be exchanged for a different marker (e.g., registration target frame 540, etc.) depending on the robot 114, the operation, and/or the type of registration to be performed.

The second registration assembly 500B, shown in FIG. 5B, may correspond to the registration assembly 300 (e.g., described in conjunction with FIGS. 3A-3D, etc.) or the registration assembly 400 (e.g., described in conjunction with FIGS. 4A-4C, etc.) with a registration target frame 540 mounted to the target receiving surface 308 of the elongate arm 304. The registration target frame 540 may correspond to a Star marker that includes marker beads 542 or other fiducials that are arranged in a pattern on at least one surface of the registration target frame 540. The registration target frame 540 may be removably attached to the target receiving surface 308 via at least one marker mount screw 544. In some examples, the registration target frame 540 may be exchanged for a different marker (e.g., registration target frame 340, etc.) depending on the robot 114, the operation, and/or the type of registration to be performed.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A robotic registration kit, comprising:
an elongate arm extending longitudinally from a proximal end to a distal end, the elongate arm comprising:
a target receiving surface disposed at the distal end; and
a robot mount bracket disposed at the proximal end and protruding from the elongate arm in a direction away from a longitudinal axis of the elongate arm, the robot mount bracket comprising a robot attachment surface disposed at a proximal end of the robot mount bracket;
a clamp screw extending a length along a rotation axis from a first end of the clamp screw to a second end of the clamp screw, the clamp screw comprising:
a bracket contact surface disposed at the first end, wherein the bracket contact surface is in contact with the robot mount bracket; and
a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis; and
a clamp, comprising:
a plate;
a threaded hole running through the plate, wherein the threaded outer surface of the clamp screw is threadedly engaged with the threaded hole; and
a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp.

2. The robotic registration kit of claim 1, further comprising:
a registration target frame mounted to the target receiving surface of the elongate arm, the registration target frame comprising marker beads that are arranged in a pattern on at least one surface of the registration target frame.

3. The robotic registration kit of claim 2, further comprising:
a sterile sleeve that fully encloses the robotic registration kit maintaining an environment inside the sterile sleeve from an environment outside the sterile sleeve.

4. The robotic registration kit of claim 3, wherein the sterile sleeve comprises a thin film bag sealed around the robotic registration kit, and wherein the sterile sleeve is waterproof and airtight.

5. The robotic registration kit of claim 4, wherein a surface of the finger is in contact with a side surface of the robot mount bracket, and wherein the contact prevents rotation of the clamp relative to the robot mount bracket as the clamp is moved between the clamped state and the unclamped state.

6. The robotic registration kit of claim 5, wherein the rotational attachment comprises a hole disposed through the robot mount bracket with a counterbore disposed on the robot attachment surface, wherein a shoulder bolt is disposed in the hole and is fastened to the clamp screw, and wherein a head of the shoulder bolt is disposed in the counterbore.

7. The robotic registration kit of claim 6, wherein the hole comprises an axis that runs parallel to the longitudinal axis of the elongate arm, and wherein the direction away from the longitudinal axis of the elongate arm is perpendicular to the longitudinal axis.

8. The robotic registration kit of claim 7, wherein the hook portion is disposed closer to the robot attachment surface when in the clamped state than when in the unclamped state.

9. The robotic registration kit of claim 1, wherein the finger comprises:
a first arm disposed on a first side of the elongate arm; and
a second arm disposed on a second side of the elongate arm, wherein the first side of the elongate arm is arranged opposite the second side of the elongate arm.

10. The robotic registration kit of claim 9, wherein the hook portion joins the first arm to the second arm.

11. The robotic registration kit of claim 9, wherein the hook portion comprises a first hook portion disposed on an end of the first arm and a second hook portion disposed on the end of the second arm, and wherein the first hook portion and the second hook portion are separate from one another.

12. A robotic system, comprising:
a robot arm comprising a plurality of links and joints running from a distal end to a proximal end;
an end-effector foot attached to the robot arm at the proximal end; and
a registration assembly attached to the end-effector foot, the registration assembly comprising:
an elongate arm extending longitudinally from a proximal arm end to a distal arm end, the elongate arm comprising:
a target receiving surface disposed at the distal arm end; and
a robot mount bracket disposed at the proximal arm end and protruding from the elongate arm in a direction away from a longitudinal axis of the elongate arm, the robot mount bracket comprising a robot attachment surface disposed at a proximal end of the robot mount bracket;
a clamp screw extending a length along a rotation axis from a first end to a second end, the clamp screw comprising:
a bracket contact surface disposed at the first end, wherein the bracket contact surface is in contact with the robot mount bracket; and
a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis; and
a clamp, comprising:
a plate;
a threaded hole running through the plate, wherein the threaded outer surface of the clamp screw is threadedly engaged with the threaded hole; and
a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp.

13. The robotic system of claim 12, wherein the clamp screw comprises a thumb wheel disposed at the second end.

14. The robotic system of claim 13, wherein the registration assembly is attached to the end-effector foot in the clamped state, and wherein the registration assembly is releasable from being attached to the end-effector foot in the unclamped state.

15. The robotic system of claim 14, wherein the registration assembly further comprises:
a registration target frame mounted to the target receiving surface of the elongate arm, the registration target frame comprising marker beads that are arranged in a pattern on at least one surface of the registration target frame.

16. The robotic system of claim 15, wherein the registration assembly further comprises:
a sterile sleeve sealed around and fully enclosing the registration assembly maintaining an environment inside the sterile sleeve from an environment outside the sterile sleeve.

17. The robotic system of claim 16, wherein the registration assembly is attached to the end-effector foot in the clamped state without penetrating the sterile sleeve.

18. The robotic system of claim 17, wherein the registration assembly is separable from the end-effector foot, in the unclamped state, while maintaining the environment inside the sterile sleeve from the environment outside the sterile sleeve.

19. The robotic system of claim 17, wherein a robot sterile sleeve covers the end-effector foot, and wherein an unpenetrated portion of the robot sterile sleeve and an unpenetrated portion of the sterile sleeve are clamped between the robot mount bracket and the end-effector foot along the robot attachment surface.

20. A sealed robotic registration assembly, comprising:
an elongate arm extending longitudinally from a proximal end to a distal end, the elongate arm comprising:
a target receiving surface disposed at the distal end; and
a robot mount bracket disposed at the proximal end and protruding from the elongate arm in a direction away from a longitudinal axis of the elongate arm, the robot mount bracket comprising a robot attachment surface disposed at a proximal end of the robot mount bracket;
a registration target frame mounted to the target receiving surface of the elongate arm, the registration target frame comprising marker beads arranged in a pattern on at least one surface of the registration target frame;
a clamp screw extending a length along a rotation axis from a first end of the clamp screw to a second end of the clamp screw, the clamp screw comprising:
a bracket contact surface disposed at the first end, wherein the bracket contact surface is in contact with the robot mount bracket; and
a threaded outer surface extending along a portion of the length, wherein the clamp screw is rotationally attached to the robot mount bracket at a rotational attachment, wherein the rotational attachment allows the clamp screw to rotate about the rotation axis, and wherein the rotational attachment prevents axial movement of the clamp screw along the rotation axis;
a clamp, comprising:
a plate;
a threaded hole running through the plate, wherein the threaded outer surface of the clamp screw is threadedly engaged with the threaded hole; and
a finger formed from the plate and extending a distance to a clamp end of the finger, the clamp end comprising a hook portion that is disposed offset proximally past the robot attachment surface of the robot mount bracket, wherein the clamp is moveable between a clamped state and an unclamped state via a rotational movement of the clamp screw relative to the clamp; and
a sterile sleeve sealed around and fully enclosing the elongate arm, the registration target frame, the clamp screw, and the clamp maintaining an environment inside the sterile sleeve from an environment outside the sterile sleeve.
